(19) United States
(12) Patent Application Publication
(10) Pub. No.: US 2016/0310067 A1
(43) Pub. Date: Oct. 27, 2016

(54) A BABY MONITORING DEVICE

(71) Applicant: KONINKLIJKE PHILIPS N.V., Eindhoven (NL)

(72) Inventors: ADRIENNE HEINRICH, EINDHOVEN (NL); KARL CATHARINA VAN BREE, EINDHOVEN (NL); VINCENT JEANNE, EINDHOVEN (NL)

(21) Appl. No.: 15/104,545

(22) PCT Filed: Dec. 17, 2014
(86) PCT No.: PCT/EP2014/078105

§ 371 Date: Jun. 15, 2016

(30) Foreign Application Priority Data
Dec. 19, 2013 (EP) 13198452.8

(51) Int. Cl. A61B 5/00; A61B 5/113; G06K 9/62; H04N 7/18; G06T 7/20; A61B 5/0205; A61B 5/11

(52) U.S. Cl. CPC A61B 5/4815; A61B 5/02055; A61B 5/113; A61B 5/1115; A61B 5/1128; A61B 5/7264; H04N 7/183; G06T 7/20; G06K 9/6267; A61B 2503/04

(57) ABSTRACT

A baby monitoring system (10) is provided which gives insight in the sleeping behaviour of a child based on the motion of the child in the bed (1). The baby monitoring system (10) comprises a video camera(11), a motion estimator (21) and a processor (22) to classify the observed motions into events. A set of events gives a parent an insight in the sleeping behaviour of the child.

The invention relates to a baby monitoring device.

### BACKGROUND OF THE INVENTION

It has been recognized that the sleep behavior of a child is of high importance to the mental and physical development of a child. Therefore, there is a growing need to obtain objective data on the sleep behaviour of children. The growing need is not only felt in medical treatments, but also by parents in daily life. Furthermore parents would like to gain insight in the sleep rhythm of their child. Unfortunately, it is not easy to obtain objective sleep related data in a non-medical environment: a parent is not always able to keep an eye on the child, when it is in bed, and, if able to keep an eye on the child, the parent is often not sufficiently alert to track the observed sleep state correctly, especially not during the nights.

In general parents find it difficult to determine how long their child has been sleeping and how their sleep behaviour and development is.

US 2007/0156060 A1 discloses an apparatus for automatically monitoring sleep, including a video recorder for recording live images of a subject sleeping, including a transmitter for transmitting the recorded images in real-time to a mobile device, and a computing device communicating with said transmitter, including a receiver for receiving the transmitted images in real-time, a processor for analyzing in real-time the received images and for automatically inferring in real-time information about the state of the subject, and a monitor for displaying in real-time the information inferred by said processor about the state of the subject.

### SUMMARY OF THE INVENTION

It is an object of the invention to provide for an objective representation of the sleep behaviour and sleep development of a child.

According to the invention this object is realized in that a baby monitoring device for monitoring a baby in a crib comprises a video camera arranged to provide a video signal for detecting a sequence of motions of the baby, an MPEG video encoder comprising a motion estimator arranged to classify the sequence of motions based on motion estimation carried out on the video signal by the MPEG video encoder during compression for classifying the sequence of motions received from the motion sensor into small amplitude motions, intermediate amplitude motions and large amplitude motions (classified motions) and a processor for classifying an event based on a sequence of small, intermediate and large amplitude motions received from the motion estimator. The video camera is arranged to detect movement of the child or baby. The MPEG video encoder comprises a motion estimator, which uses the movements detected by the video camera to classify the sequence of motions based on motion estimation carried out on the video signal by the MPEG video encoder during compression to classify the movements as small amplitude motions, intermediate amplitude motions or large amplitude motions. The motion estimator distinguishes between the several classified motions based on the amplitude of the motion. The motion amplitudes can be easily extracted from the MPEG video encoder during compression of the video signal as the motion estimator in an MPEG video encoder calculates motion vectors. From these motion vectors only the motion amplitudes or classified motions need to be stored for the purpose of the invention, not the direction of the motion vectors as normally also obtained by the motion estimator during MPEG video encoding. The classified motion classified by the motion estimator will subsequently be fed to the processor for classifying a sequence of small, intermediate and large amplitude motions as an event. An event is an interpretation of the processor of the sleep behaviour of the child. By measuring and analyzing the movement of a child, information on the sleep behaviour of the child can be obtained.

An advantageous embodiment of the invention is that the motion estimator may classify breathing by the baby as a small amplitude motion, a movement of the body of the baby within the crib as intermediate movement and a movement of the body of the baby in or out of the crib as a large amplitude motion. The classification in small, intermediate and large motion gives a parent insight in the sleeping behaviour of their child. Movement of the chest, i.e. breathing, may be classified as a small amplitude motion. A small amplitude motion may represent quiet sleep, because body movement is not detected by the motion sensor. An intermediate amplitude motion may represent active sleep or alertness. The alertness may include vocalization. Breathing motion is present, but is obscured by movement of the body. A large amplitude motion may represent a parent taking the baby out of bed or putting the baby into bed. Small and intermediate amplitude motions are obscured/overruled by the large amplitude motions. For clarity sake, if no motion is detected, then the motion estimator classifies an absence of motion.

In a preferred embodiment the baby monitoring system comprises a sound sensor and the processor classifies an event on sound received from the sound sensor as well. A sound sensor, next to the motion sensor, enables the system to monitor sound additionally to motion. The sound sensor provides additional input to the processor. The processor consequently classifies an event based on a sequence of small, intermediate and large amplitude motions received from the motion estimator and on sound received from the sound sensor. The baby monitoring system comprising only a motion sensor is able to distinguish the baby's behaviour in bed between classified motions, so that the system determines whether the baby is lying quietly or moving. The dual input of the processor enables the baby monitoring system to distinguish between the five behavioral states Quiet Sleep, Active Sleep, Quiet Alertness, Active Alertness and Crying. The presence of an additional sensor, such as a sound sensor, thus enables the system to monitor more reliably the sleep behaviour of a child.

Preferably the processor is arranged to use changes of other vital signs to determine the event. Other vital signs may include for example heart rate or body temperature. The additional information provided by the input of other vital signs provides for a more reliable baby monitoring system. By making use of the additional data incorrect analysis of data from the motion sensor and/or false alarms can be prevented. For example, when the motion sensor does not detect motion, the baby is either in bed and not breathing or the baby is out of bed. In the first situation an immediate response of the parent is required and therefore the parent should be alerted, while in the second situation there is no need to alert the parent. Additional information from the vital signs, such as body temperature, may be used to determine whether an alarm should be provided or not. When no body temperature or a temperature in the range of the environment is detected, the processor may be adapted not provide an alert, as it is probable that the baby is not present in the bed. If, however, a temperature is measured at normal body temperature or higher or lower, but well above the environmental temperature, the processor may trigger an alarm. In this situation a child is probably present in the bed, either in hyperthermia or having a fever, and not breathing. By arranging the processor to use both data from the motion sensor and from a vital signs sensor the event can be determined more accurately and the number of false interpretation can be reduced.

In a preferred embodiment the processor is arranged to classify a sequence of a small amplitude motion followed by an intermediate amplitude motion followed by a small amplitude motion as a baby in bed and restless movement event. The order of the classified motions indicate that the baby was lying quietly and that only motion of the chest was observed, followed by body movement and again motion of the chest. The baby is most likely sleeping quietly or alert quietly, followed by active sleeping or active alert and again sleeping quietly or alert quietly. This provides the parent with information that the baby is in bed and sleeping restless.

In another preferred embodiment the processor is arranged to classify a sequence of an absence of motion followed by a large amplitude motion followed by a small amplitude motion or an intermediate amplitude motion as a baby is put to bed event. The order of the motion amplitudes indicate that first there was no motion, followed by a motion larger than the baby can make and finally a motion of the chest, indicating breathing. This provides the parent with information that the baby is put to bed and that he is lying quietly, either sleeping or alert and does not need immediate attention.

In a further preferred embodiment the processor is arranged to classify a sequence of a small amplitude motion or an intermediate amplitude motion followed by a large amplitude motion followed by absence of motion as a baby is taken out of bed event.

The order of the motion amplitudes indicate that the baby was first quietly lying in bed and that he started moving with his body, such as waving or turning around. After that the baby was taken out of bed, as the large amplitude motion indicates a motion larger than a baby can make itself, such as a parent taking the baby out of bed.

In another preferred embodiment the processor is arranged to classify a sequence of small amplitude motions as a baby in bed event. A sequence of small motion amplitudes indicates that only breathing is observed and that larger body movements are not observed. The processor indicates this data sequence as that the baby is in bed and quietly sleeping or awake. This is comforting information for the parent and does not require an alert to the parent.

In a further preferred embodiment the processor is arranged to classify a sequence of an intermediate amplitude motion followed by another intermediate amplitude motion as a baby awake in bed event. A continuous order of intermediate amplitude motions representing body motion is an indication that the baby is awake in bed. This may be a signal for the parent to go and see the baby.

Advantageously the processor is arranged to provide statistics based on a sequence of classified events. The classified events based on the classification of sequences of classified motions may, next to real-time data representation, be used to determine the sleep behaviour of a child over a longer period. It can for example be used to determine how long the baby sleeps during the day or night, how long certain behavioral states take or to predict the optimal sleeping time and time to wake up the baby. It can also be used by other caretakers to compare the data of a child with a group of children of the same age. This is beneficial, when the baby is presumed to sleep too little or when the baby develops slower than expected.

In another preferred embodiment the processor is arranged to provide statistics based on a sequence of classified motions. Provide statistics based on classified motions is helpful if the baby wakes up too often compared to other children of the same age or if the baby develops not well. Too many or too long time intervals classified as intermediate amplitude motion and too few or too short time intervals classified as small amplitude motion indicate that the baby is often sleeping actively or actively awake and that it does not often sleep quietly. Quiet sleep or deep sleep is associated with processing information that is associated with learning and is therefore necessary for a healthy development.

In another embodiment the baby monitoring system is arranged to log events. The logging of events provides information to the parent on the sleeping behaviour of the child. The log shows the sequence of events during a period of time, for example a period of 24 hours. It gives the parent objective feedback on how the baby slept in the period.

### BRIEF DESCRIPTION OF THE DRAWINGS

These and other aspects of the pacifier of the invention will be further elucidated and described with reference to the drawings in which

FIG. 1 illustrates a schematic drawing of the set-up according to an embodiment,

FIG. 2 shows a photo image overlaid with motion vectors,

FIG. 3 shows a flowchart exemplarily illustrating an embodiment of a method for classifying events,

FIG. 4 shows a graph exemplarily for a few sequences of motion.

### DETAILED DESCRIPTION OF THE DRAWINGS

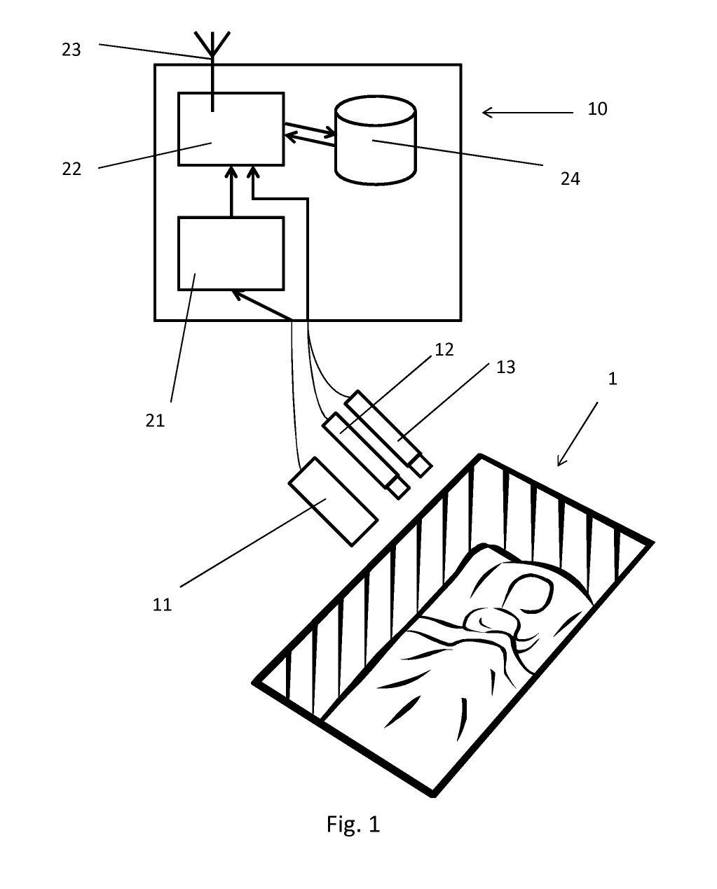

FIG. 1 shows schematically a baby monitoring system 10 according to the invention. The system 10 comprises a motion sensor 11, such as a video camera, a motion estimator 21 and a processor 22. The baby monitoring system 10 can be equipped with an additional sensor for recording sound, a sound sensor 12, and/or with an additional sensor for detecting vital signs, such as heart rate or pulsation, a vital signs sensor 13. The baby monitoring system can also be equipped with a data storage 24. The functions of the invention can be integrated or embedded in a common baby monitoring system 10, which records sound and video of the baby in the bed 1 and provides it realtime to the parent, or can be provided in a baby monitoring system 10 suited for the analysis of sleep behaviour of the invention.

The object of the baby monitoring system 10 is to monitor a child in a bed 1 and to provide information on the sleep behaviour of the child. The motion sensor 11 is arranged for detecting a sequence of images of the baby in the bed 1. The motion estimator 21 uses the images detected by the motion sensor 11 to calculate a motion amplitude from two subsequent images and classifies the motion amplitudes/movements as small amplitude motions, intermediate amplitude motions or large amplitude motions. The classified motions as classified by the motion estimator 21 are fed to the processor 22 for classifying a sequence of small, intermediate and large amplitude motions as an event. An event is an interpretation of the processor 22 of the sleep behaviour of the child. By measuring and analyzing the movement of the child in and into and out of the bed 1, information on the sleep behaviour of the child can be obtained.

The sound sensor 12, next to the motion sensor 11, enables the system to monitor sound in addition to motion. The sound sensor 12 provides additional input to the processor 22. The processor 22 consequently classifies an event based on a sequence of small, intermediate and large amplitude motions received from the motion estimator and on sound received from the sound sensor.

The appliance of a vital signs sensor 13 provides additional information for a more reliable baby monitoring system. The vital signs sensor 13 can be a separate sensor, but the vital signs can also be monitored by the motion sensor 11. By making use of the additional data incorrect analysis of data from the motion sensor and/or false alarms can be prevented.

The processor 22 comprises an antenna 23 for communicating data, realtime or stored, to a receiving unit (not shown). The receiving unit (not shown) is generally located outside the room of the baby (not shown), for example a parent unit or a smartphone, so that a person outside the room, for example the parent of the child, can look after the child.

The processor 22 transfers the classified motions and classified events to the data storage 24 to create a log of the history of classified motions. For each time period at least the largest classified motion detected during that time period is stored.

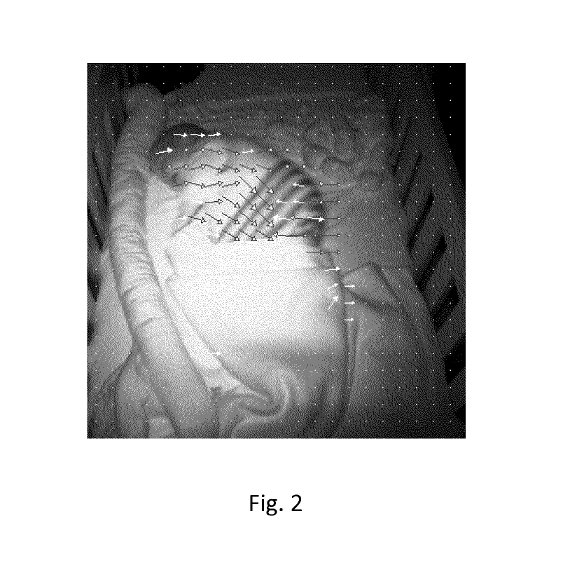

FIG. 2 shows a photo overlaid with motion amplitudes/motion vectors. The motion vectors are calculated by the motion estimator 21 using common MPEG video encoding techniques and represent a visual interpretation of motion in the course of time. The larger the motion vector, the larger the movement. Calculation of the motion amplitude is a well-known video processing process and will not further be elucidated here. For regular video processing both motion amplitude and direction are relevant but for baby monitoring only the amplitude of the motion needs to be determined.

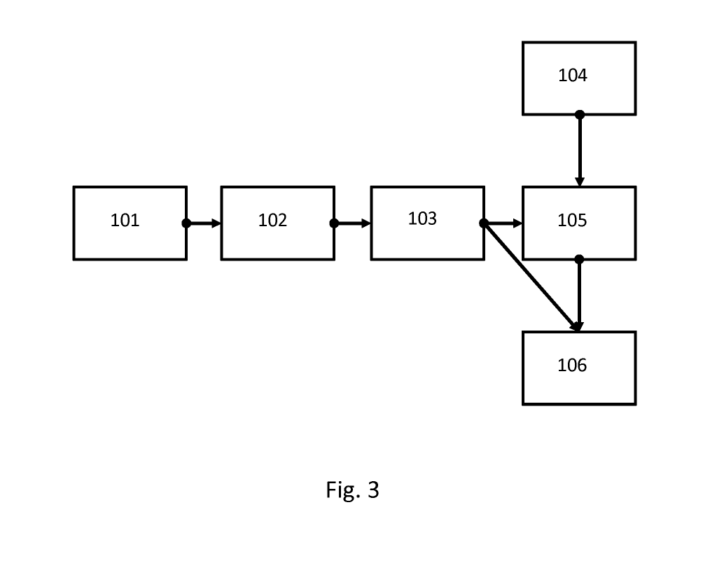

FIG. 3 schematically shows a flowchart of the method to classify events. In step 101 an image of a baby in the bed 1 is recorded. Step 101 is performed by the motion sensor 11.

In step 102 a motion amplitude is calculated from two subsequent images. In this step the size and the direction of a motion are determined. The motion amplitude comprises the size of the motion.

In step 103 the motion amplitude from step 102 is classified into classified motions. Three different classifications are distinguished: small amplitude motion, intermediate amplitude motion and large amplitude motion. The motion estimator 21 classifies breathing by the baby as a small amplitude motion, a movement of the body of the baby within the crib as intermediate movement and a movement of the body of the baby in or out of the crib as a large amplitude motion. The classification in small, intermediate and large motion gives a parent insight in the sleeping behaviour of their child. Movement of the chest, i.e. breathing, is classified as a small amplitude motion. A small amplitude motion represents quiet sleep, because body movement is not detected by the motion sensor. An intermediate amplitude motion represents active sleep or alertness. The alertness may include vocalization. Breathing motion is present, but is obscured by movement of the body. A large amplitude motion represents a parent taking the baby out of bed or putting the baby into bed. Small and intermediate amplitude motions are obscured/overruled by the large amplitude motions. For clarity sake, if no motion is detected, then the motion estimator classifies an absence of motion.

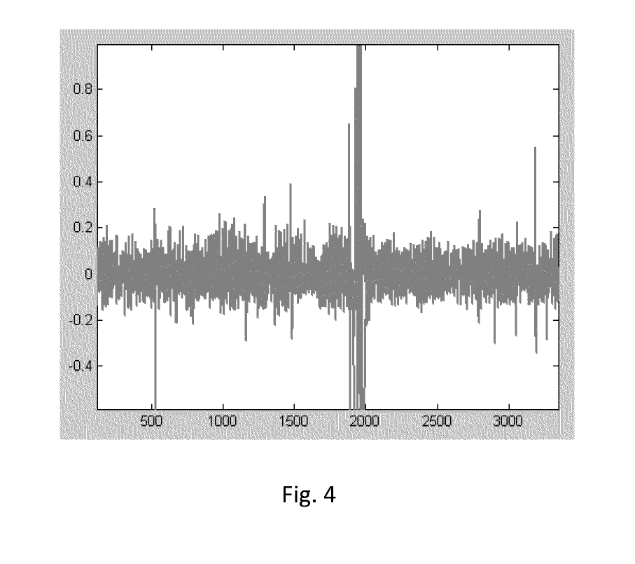

An example of a sequence of motion amplitudes is shown in FIG. 4. The sequence of motion amplitudes is calculated using common MPEG video encoding techniques for motion analysis. An example of a sequence of motion amplitudes is shown in FIG. 4. The sequence of motion amplitudes is calculated using common MPEG video encoding techniques for motion analysis. An MPEG video encoder comprises a motion estimator which is arranged to classify the sequence of motions based on motion estimation carried out on the video signal by the MPEG video encoder during compression. The motion amplitudes can be easily extracted from the MPEG Video encoder during compression of the video signal as the motion estimator in an MPEG video encoder calculates motion vectors. From these motion vectors only the motion amplitudes or classified motions need to be stored for the purpose of the invention, not the direction of the motion vectors as normally also obtained by the motion estimator during MPEG video encoding. On the horizontal axis the time is plotted. The motion amplitude is plotted on the vertical axis. During the measurement the motion amplitude is generally between −0.2 and 0.2. This motion amplitude represents a small motion amplitude and will be classified by the processor 22 as a small amplitude motion. The small amplitude motion is valued as breathing motion. Around 2000 on the horizontal axis a number of large motion amplitudes is observed. These large motion amplitudes will be classified by the processor 22 as a large amplitude motion. The large amplitude motion will be valued as a motion from inside the bed 1 to the outside or vice versa. The other motion amplitudes will be classified as intermediate amplitude motions. The intermediate amplitude motions will be valued as a movement of a baby in the bed.

Dependent on the sensitivity settings of the processor 22 the single time frame intermediate amplitude motions can be ignored or will be logged in the data storage 24. The processor 22 will classify the order of these subsequent classified motions as a baby in bed event, followed by an interference of a parent, followed by a baby in bed event. The parent may for example have come to the baby's bed 1 to cover the baby with a blanket or remove a subject from the baby's face.

Step 102 and 103 are performed by the motion estimator. Classified motions are input for step 105 and for step 106.

The classified motions are processed to step 105. In step 105 the processor 22 receives a sequence of classified motions and subsequently classifies an event bases on a number of subsequent classified motions. The processor 22 will for example classify a sequence of a small amplitude motion followed by an intermediate amplitude motion, followed by a small amplitude motion as a baby in bed and restless movement event. The order of the classified motions indicate that the baby was lying quietly and that only motion of the chest was observed, followed by body movement and again motion of the chest. The baby is most likely sleeping quietly or alert quietly, followed by active sleeping or active alert and again sleeping quietly or alert quietly. This provides the parent with information that the baby is in bed and sleeping restless. Another example is a sequence of an absence of motion followed by a large amplitude motion followed by a small amplitude motion or an intermediate amplitude motion and will be classified by the processor 22 as a baby is put to bed event. The order of the motion amplitudes indicate that first there was no motion, followed by a motion larger than the baby can make and finally a motion of the chest, indicating breathing. This provides the parent with information that the baby is put to bed and that he is lying quietly, either sleeping or alert and does not need immediate attention.

Step 105 may receive additional input from step 104. In step 104 sound is recorded near the child by the sound sensor 12 and is sent to the processor 22. In step 105 the processor 22 classifies an event based on a sequence of small, intermediate and large amplitude motions received from the motion estimator 22 and on sound received from the sound sensor 12. The baby monitoring system 10 comprising only a motion sensor 11 is able to distinguish the baby's behaviour in bed 1 between classified motions, so that the system 10 determines whether the baby is lying quietly or moving. The dual input of the processor 22 enables the baby monitoring system 10 to distinguish between the five behavioral states Quiet Sleep, Active Sleep, Quiet Alertness, Active Alertness and Crying. The presence of an additional sensor, such as a sound sensor 12, thus enables the system to monitor more reliably the sleep behaviour of a child. Classified events will be sent to the data storage 24.

The data, classified events from step 105 and classified motions from step 103, will be stored in the data storage 24 in step 106. The classified motions are available for classifying an event based on a sequence of classified motions. The classified motions and the classified events are available to give the parent insight in the sleep behaviour of the child in the bed (1). It provides the parent with objective feedback on how the baby slept. Instead of storing the classified motions, one can store the sequence of motion amplitudes, i.e. instead of sequence of classified motions that represent the average or largest motion amplitudes encountered during each time period one stores the measured motion amplitudes.

1. A baby monitoring device for monitoring a baby in a crib, comprising:
a video camera arranged to provide a video signal for detecting a sequence of motions of the baby,
a motion estimator for classifying the sequence of motions received from the video camera,
a processor for classifying an event based on a sequence of small, intermediate and large amplitude motions received from the motion estimator,
wherein the baby monitoring device comprises an MPEG video encoder comprising the motion estimator and where the motion estimator is arranged to classify the sequence of motions received from the video camera into small amplitude motions, intermediate amplitude motions and large amplitude motions based on motion estimation carried out on the video signal by the MPEG video encoder during compression.

2. Baby monitoring device as claimed in claim 1 where motion estimator classifies breathing by the baby as a small amplitude motion, a movement of the body of the baby within the crib as an intermediate amplitude motion and a movement of the body of the baby in or out of the crib as a large amplitude motion.

3. A baby monitoring device as claimed in claim 2, wherein the baby monitoring system comprises a sound sensor and wherein the processor classifies an event based on sound received from the sound sensor and on a sequence of small, intermediate and large amplitude motions received from the motion estimator.

4. A baby monitoring device as claimed in claim 2 where the processor is arranged to use changes of other vital signs to determine the event.

5. A baby monitoring device as claimed in claim 1, where the processor is arranged to classify a sequence of a small amplitude motion followed by a intermediate amplitude motion followed by a small amplitude motion as a baby in bed and restless movement event.

6. A baby monitoring device as claimed in claim 1, where the processor is arranged to classify a sequence of a absence of motion followed by a large amplitude motion followed by a small amplitude motion or an intermediate amplitude motion as a baby is put to bed event.

7. A baby monitoring device as claimed in claim 1, where the processor is arranged to classify a sequence of a small amplitude motion or an intermediate amplitude motion followed by a large amplitude motion followed by absence of motion as a baby is taken out of bed event.

8. A baby monitoring device as claimed in claim 1, where the processor is arranged to classify a sequence of small amplitude motion and intermediate amplitude motions as a baby in bed event.

9. A baby monitoring device as claimed in claim 1, where the processor is arranged to classify a sequence of an intermediate amplitude motion followed by another intermediate amplitude motion as a baby awake in bed event.

10. A baby monitoring device as claimed in claim 1 where the processor is arranged to provide statistics based on a sequence of classified events.

11. A baby monitoring device according to claim 1 wherein the processor is arranged to provide statistics based on a sequence of classified motions.

12. A baby monitoring device according to claim 1 wherein the processor is arranged to log events.
